(19) United States
(12) Patent Application Publication
(10) Pub. No.: US 2022/0018829 A1
(43) Pub. Date: Jan. 20, 2022

(54) Methods and Compositions for Clinical Evaluation of Therapeutic Agents

(71) Applicant: Janssen Biotech, Inc., Horsham, PA (US)

(72) Inventors: Esi Sama Natya Lamouse-Smith, New York, NY (US); Nina Sabins, Plymouth Meeting, PA (US)

(21) Appl. No.: 17/428,831

(22) PCT Filed: Jan. 15, 2020
(86) PCT No.: PCT/IB2020/050303

§ 371 Date: Aug. 5, 2021

Related U.S. Application Data
(60) Provisional application No. 62/802,732, filed on Feb. 8, 2019.

(51) Int. Cl. G01N 33/50

(52) U.S. Cl. CPC G01N 33/5088; G01N 2500/10

(57) ABSTRACT

Methods and kits are described for clinical evaluation of a therapeutic agent. In particular, an immune perturbation reflecting an immune response signature of a disease is induced in a healthy subject, and a therapeutic agent of interest is clinically evaluated in the healthy subject with the immune perturbation.

### TECHNICAL FIELD

The disclosure relates to the field of systems approach to immune responses and treatment effects, particularly methods of clinical evaluation of a therapeutic agent in a healthy subject with an induced immune perturbation comprising an immune response signature of a disease of interest.

### BACKGROUND

Currently, most first-in-human studies are intended to evaluate safety and pharmacokinetics of new drugs with less emphasis on pharmacodynamic drug effects. In healthy human volunteers many of the disease specific molecules are not expressed due to lack of an acute or chronic (e.g., autoimmune) immune stimulus, therefore, the impact of drugs targeting these molecules cannot be easily measured.

Vaccines have been included in early clinical studies of a therapeutic agent. For example, in a clinical study on vedolizumab (an α4β7 integrin monoclonal antibody), healthy participants received a single intravenous dose of vedolizumab, followed by intramuscularly administered HBV vaccine and orally administered cholera vaccine (Wyant et al., Gut. 2015 January; 64(1):77-83). It was shown that vedolizumab did not alter the response to intramuscularly administered HBV antigen but reduced the response to the orally administered cholera antigen, demonstrating its gut-selective mechanism of action. However, the study did not show whether the HBV vaccine or cholera vaccine had induced an immune perturbation containing an immune response signature reflective of moderate-to-severe active ulcerative colitis, the disease targeted by vedolizumab. Although antigenic challenges exist that are capable of inducing activation of immunological targets, understanding these biological responses in the context of a clinical trial setting remains poorly understood.

Limitations exist in current methods of evaluating disease pathology and drug mechanism at the cellular level. For example, existing methods to evaluate immune status rely on qualitative or quantitative measurements of immunoglobulins (e.g., total IgG, IgM, IgA) and relative proportions of general immune cell populations (e.g., CD4, CD8, neutrophils, monocytes) without regard to functional status or specificity. Biomarkers measuring immunological changes in past studies have largely been restricted to assays of soluble proinflammatory factors and gene expression; whereas phenotypic and functional characterization of immune cell populations have been limited. An extended analysis that incorporates features of functional immune response parameters could be part of an essential strategy toward understanding immune-mediated pathogenic processes and measuring the effects of different immune modulatory treatment modalities. However, measurement of immune cell profiles are not currently standardized for use in clinical studies.

There is a need of new technologies and approaches to enable multidimensional analysis of immune responses that will strengthen precision medicine approaches in clinical studies.

### SUMMARY OF THE INVENTION

According to embodiments of the application, an immune perturbation comprising an immune response signature of a disease is induced in a healthy subject, and a therapeutic agent of interest is clinically evaluated in the healthy subject with the immune perturbation.

In one general aspect, the application is related to a method of identifying an antigen that elicits an immune perturbation in a healthy subject reflecting an immune response signature of a disease, the method comprising:
(1) administering to the healthy subject an effective amount of a composition comprising an antigen;
(2) collecting a biological sample from the healthy subject before and at multiple time points after the administering of step (1);
(3) measuring longitudinal changes from baseline (i.e., before the administering of step (1)) in immune cell populations and/or inflammatory mediators in the biological sample;
(4) comparing the measured longitudinal changes with the immune response signature of the disease to detect an effect of an immune perturbation induced by the antigen in the healthy subject; and
(5) identifying the antigen that elicits the immune perturbation in the healthy subject reflecting the immune response signature of the disease.

In another general aspect, the application is related to a method of predicting an effect of a therapeutic agent, the method comprising:
(1) administering to a healthy subject an effective amount of a composition comprising an antigen known to elicit an immune perturbation in the healthy subject reflecting an immune response signature of a disease;
(2) administering a pharmaceutical composition comprising the therapeutic agent to the healthy subject having the immune perturbation;
(3) measuring an impact of the therapeutic agent on the immune perturbation in the healthy subject; and
(4) predicting the effect of the therapeutic agent in a subject in need of a treatment of the disease based on the impact measured in (3).

In one embodiment of the application, the method further comprises:
(5) administering the pharmaceutical composition comprising the therapeutic agent to the subject in need of the treatment of the disease, and
(6) evaluating the effect of the therapeutic agent in the subject in need of the treatment of the disease.

In one general aspect, the application is related to another method of predicting an effect of a therapeutic agent, the method comprising:
(1) administering to a subject in need of a treatment of a disease an effective amount of a composition comprising an antigen known to elicit an immune perturbation in a healthy subject reflecting an immune response signature of a disease;
(2) collecting a biological sample from the subject in need of the treatment before and at multiple time points after the administering of step (1);
(3) measuring longitudinal changes from baseline (i.e., before the administering of step (1)) in immune cell populations and/or inflammatory mediators in the biological sample;
(4) comparing the measured longitudinal changes with the immune response signature of the disease to detect an effect of an immune perturbation induced by the antigen in the subject in need of the treatment; and
(5) predicting the effect of the therapeutic agent in the subject in need of the treatment based on the immune perturbation induced by the antigen.

In one embodiment of the application, the method further comprises:
(6) administering a pharmaceutical composition comprising the therapeutic agent to the subject in need of the treatment of the disease, and
(7) evaluating the effect of the therapeutic agent in the subject in need of the treatment of the disease.

In another embodiment of the application, the method further comprises
(8) measuring an impact of the therapeutic agent on the immune perturbation induced by the antigen in the subject in need of the treatment to thereby evaluate the effect of the therapeutic agent in the subject.

In one embodiment, a method of the application is used in a first-in-human clinical evaluation of a therapeutic agent for the treatment of a disease.

In one embodiment, the effect of a therapeutic agent on a subject, preferably a human subject in need of a treatment of a disease, comprises a pharmacodynamic effect, including but not limited to a biochemical, physiologic, and/or molecular effect of therapeutic agent on the subject. Preferably, the effect comprises a clinical pharmacodynamic effect selected from at least one of clinical efficacy and safety of the therapeutic agent in the subject. The effect can also comprise pharmacokinetics of the therapeutic agent in the subject.

Any suitable antigen can be used in the invention to induce a desirable immune perturbation. Preferably, the antigen is a vaccine previously approved for use in human. According to an embodiment of the application, the antigen is selected from the group consisting of a smallpox vaccine (e.g., Imvamune®/Imvanex®), a shingles vaccine (e.g., Shingrix®), a zoster vaccine (e.g., Zostavax®), a tetanus vaccine, LPS endotoxin, and an antigenic skin challenge (such as CANDINO, Candida albicans).

Other aspects, features and advantages of the invention will be apparent from the following disclosure, including the detailed description of the invention and its preferred embodiments and the appended claims.

### BRIEF DESCRIPTION OF THE DRAWINGS

The foregoing summary, as well as the following detailed description of the invention, will be better understood when read in conjunction with the appended drawings. It should be understood that the invention is not limited to the precise embodiments shown in the drawings.

In the drawings:

FIG. 1 shows that vaccines can highlight immune targets/pathways of disease pathogenesis targeted for drug modulation; and

FIG. 2 shows the study design of a phase 0 clinical study to provide multi-level analysis of immune responses in healthy human subjects administered with known vaccines.

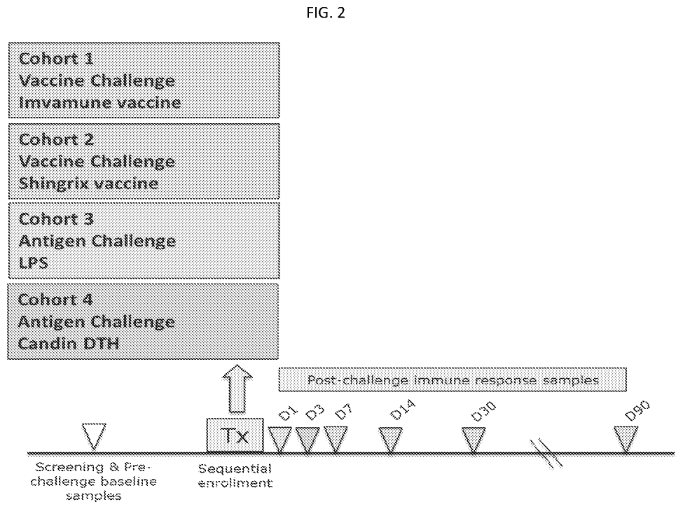

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the present invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set in the specification. All patents, published patent applications and publications cited herein are incorporated by reference as if set forth fully herein. It must be noted that as used herein and in the appended claims, the singular forms “a,” “an,” and “the” include plural reference unless the context clearly dictates otherwise.

Unless otherwise stated, any numerical value, such as a concentration or a concentration range described herein, are to be understood as being modified in all instances by the term “about.” Thus, a numerical value typically includes ±10% of the recited value. For example, a dosage of 100 μg includes 90 μg to 110 μg. As used herein, the use of a numerical range expressly includes all possible subranges, all individual numerical values within that range, including integers within such ranges and fractions of the values unless the context clearly indicates otherwise.

Throughout this specification and the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps. When used herein the term “comprising” can be substituted with the term “containing” or “including” or sometimes when used herein with the term “having.”

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising,” “containing,” “including,” and “having,” whenever used herein in the context of an aspect or embodiment of the invention can be replaced with the term “consisting of or” consisting essentially of to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, “subject” means any animal, preferably a mammal, most preferably a human, to whom will be or has been administered immunogenic components and/or compositions according to the invention. The term “mammal” as used herein, encompasses any mammal. Examples of mammals include, but are not limited to, cows, horses, sheep, pigs, cats, dogs, mice, rats, rabbits, guinea pigs, non-human primates (NHPs) such as monkeys or apes, and humans, etc., more preferably a human.

As used herein, “an effective amount” means an amount of an antigen sufficient to induce a desired immune effect or immune response in a subject in need thereof. An effective amount can vary depending upon a variety of factors. For example, with respect to “an effective amount” of a composition comprising an antigen used to induce an immune perturbation reflecting an immune response signature of a disease in a healthy subject, the effective amount can vary depending on factors such as the physical conditions of the subject, age, weight, health, etc.; the particular composition comprising the antigen used; the particular disease desired which immune response signature is reflected by the immune perturbation in the healthy subject, etc. An effective amount can be determined by one of ordinary skill in the art in view of the present disclosure.

In a healthy subject, the body maintains homeostasis of the immune system that allows the maintenance and regulation of the stability and constancy needed to keep a normal immune balance. A vaccination induces an immune perturbation, such as a primary immune response that is slow to build up and not very strong. Upon the vaccination, some of the B lymphocytes detect the antigen in the vaccine and multiply to produce more clones able to secrete particular antibodies against the antigen. Thus, vaccination increases the levels of circulating antibodies against the antigen. This primary response takes a couple weeks to build to maximum intensity. Over time, the primary response induced by the vaccine will gradually disappear, however, memory cells remain after the vaccination. Upon infection by a pathogen, a secondary response is induced, which is stronger and more rapid than the primary response. The responses resolve with time.

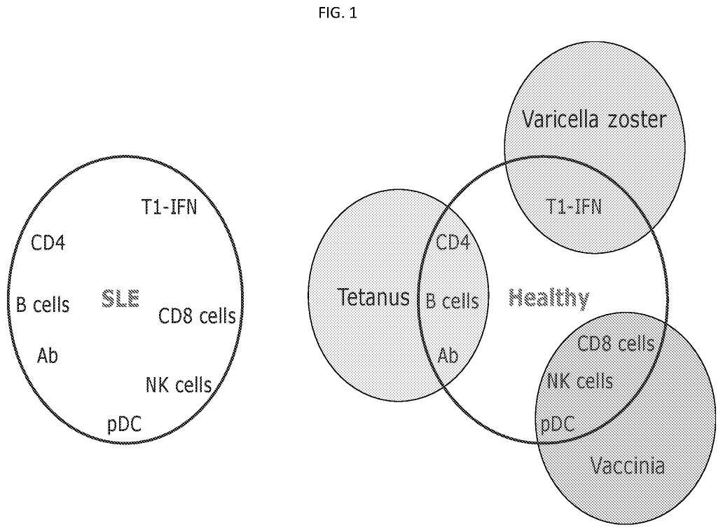

As shown in FIG. 1, vaccines can highlight immune targets and/or pathways of disease pathogenesis targeted for drug modulation. For example, systemic lupus erythematosus (SLE) is known to be associated with dysregulation and/or abnormal activity or function of various immune targets and/or pathways, such as CD4⁺ T cells, B cells, antibody (Ab) responses, plasmacytoid dendritic cells (pDCs), NK cells, CD8⁺ T cells, type I interferons (T1-IFNs), etc. Vaccines are known to induce immune perturbations of the various immune targets and/or pathways associated with SLE, thus can be used to induce an immune perturbation in a healthy subject that reflects one or more immune response signatures of SLE. For example, a Varicella zoster vaccine can be used to induce an immune perturbation in the healthy subject reflecting an immune response signature of SLE that includes the level or activity of T1-IFN, while a vaccinia vaccine can be used to induce an immune perturbation in the healthy subject reflecting an immune response signature of SLE including properties of one or more of pDCs, NK cells and CD8⁺ T cells. In addition, a tetanus vaccine can be used to induce an immune perturbation in the healthy subject reflecting an immune response signature of SLE including properties of one or more of CD4⁺ T cells, B cells, and Ab responses. In a clinical evaluation of a therapeutic agent for the treatment of SLE, the effect of the therapeutic agent on one or more immune perturbations induced by the vaccines in a healthy subject can be investigated. Results from clinical studies of the therapeutic agent in the healthy subject having the one or more immune perturbations induced by the vaccines can guide clinical studies of the therapeutic agent in a subject in need of a treatment of SLE.

As used herein, an “immune perturbation” refers to a response of the immune system of a subject induced by the administration of a composition comprising an antigen, or a change in the immune system induced by or associated with a disease in a subject. For example, an immune perturbation can comprise a primary response induced by the administration of an antigen. An immune perturbation can also comprise a change in the immune system induced by inflammation, such as chronic inflammation, associated with a disease. An immune perturbation can further comprise a change in one or more immune pathways that are dysregulated in a subject having the disease

According to embodiments of the application, an immune perturbation in a healthy subject induced by the administration of an antigen can reflect an immune response signature of a disease.

As used herein, an “immune response signature” refers to a cellular and/or molecular signature of innate and/or adaptive immunity during effector and/or memory stages of immune responses following administration of an antigen to a subject or associated with a disease in a patient. More than one immune response signatures can be induced by one antigen or associated with one disease.

As used herein, an “immune response signature of a disease” refers to an immune response signature associated with the disease observed in a patient suffering from the disease or a subject in need of a treatment of the disease. For example, an immune response signature of a disease can be defined by one or more of (1) an immune response that occurs during acute infection or chronic inflammatory conditions associated with the disease in a patient; or (2) localized and/or systemic soluble and cellular immune responses associated with the disease in a patient.

As used herein, “an immune perturbation in a healthy subject reflecting an immune response signature of a disease” refers to an immune perturbation in the healthy subject comprising an immune response signature that corresponds to an immune response signature of the disease. An immune perturbation in the healthy subject can be induced by the administration of an antigen, such as a known vaccine. An immune perturbation reflecting an immune response signature of a disease can encompass the entire duration of the immune perturbation that define an immune response signature corresponding to an immune response signature of the disease, e.g., until during which such induced immune responses have returned to near or substantially baseline levels. According to embodiments of the application, an immune perturbation induced by an antigen in a healthy subject can reflect an immune response signature of one or more diseases. In other embodiments of the application, a disease can have one or more immune response signatures. One or more antigens or vaccines can be used to induce an immune perturbation in a healthy subject reflecting one or more immune response signatures of one or more diseases.

An antigen that elicits an immune perturbation in a healthy subject reflecting an immune response signature of a disease can be identified using a method of the invention, comprising:
(1) administering to the healthy subject an effective amount of a composition comprising the antigen;
(2) collecting a biological sample from the healthy subject before and at multiple time points after the administering of step (1);
(3) measuring longitudinal changes from baseline (i.e., before the administering of step (1)) in immune cell populations and/or inflammatory mediators in the biological sample;
(4) comparing the measured longitudinal changes with the immune response signature of the disease to detect an effect of an immune perturbation induced by the antigen in the healthy subject; and
(5) identifying the antigen that elicits the immune perturbation in the healthy subject reflecting the immune response signature of the disease.

Upon administration, a therapeutic agent can result in a change in an immune perturbation induced by an antigen or a disease. For example, an immune perturbation induced by an antigen, such as a known vaccine, in a healthy subject reflecting an immune response signature of a disease can be modified upon administration of a therapeutic agent for treating the disease. An effect of a therapeutic agent can be predicted or evaluated based on its impact on the immune perturbation induced by the antigen in the healthy subject. For example, if the therapeutic agent results in shortening of the duration of the response of the immune perturbation, reduction in the amplitude of the response of the immune perturbation, and/or otherwise enhanced resolution or response of the immune perturbation, the changes can indicate or predict that the therapeutic agent is effective for the treatment of the disease in a patient in need of a treatment of the disease. Alternatively, if the therapeutic agent has no or negative impact on the resolution or response of the induced immune perturbation in the healthy subject, it can suggest that the therapeutic agent is ineffective against the disease in a patient.

Thus, in another general aspect, the application is related to a method of predicting an effect of a therapeutic agent based on a study with a healthy subject. The method comprises:
(1) administering to a healthy subject an effective amount of a composition comprising an antigen known to elicit an immune perturbation in the healthy subject reflecting an immune response signature of a disease;
(2) administering a pharmaceutical composition comprising the therapeutic agent to the healthy subject having the immune perturbation;
(3) measuring an impact of the therapeutic agent on the immune perturbation in the healthy subject; and
(4) predicting the effect of the therapeutic agent in a subject in need of a treatment of the disease based on the impact measured in (3).

According to an embodiment of the application, the method comprises administering different amounts of the pharmaceutical composition comprising the therapeutic agent to the healthy subject having the immune perturbation to thereby obtain a dosage-response relationship of the therapeutic agent.

A therapeutic agent that is predicted to be effective against a disease based on a study with a healthy subject can be further evaluated in a subject in need of a treatment of a disease. Thus, in one embodiment of the application, the method further comprises:
(1) administering the pharmaceutical composition comprising the therapeutic agent to a subject in need of a treatment of the disease, and
(2) evaluating the effect of the therapeutic agent in the subject.

According to an embodiment of the application, the method comprises administering different amounts of the pharmaceutical composition comprising the therapeutic agent to the subject in need of the treatment to obtain a dosage-response relationship of the therapeutic agent in the subject. Preferably, the different amounts of the pharmaceutical composition are selected based on a dosage-response relationship of the therapeutic agent in a healthy subject obtained using a method according to an embodiment of the application.

A therapeutic agent can target or modulate immune pathway(s) involved in one or more immune perturbations induced by or associated with a disease. An effect of a therapeutic agent can be evaluated based on its regulation of the immune pathway(s). For example, if an immune pathway is constitutively down regulated in a patient, the patient may not be responsive to a treatment with a therapeutic agent that targets an upregulated immune pathway. A subject's responsiveness to an antigen known to elicit an immune perturbation in a healthy subject reflecting an immune response signature of the disease can be used to predict the subject's responsiveness to a treatment with a therapeutic agent targeting the immune pathway(s) involved in the immune perturbation. For example, if the antigen induced an immune perturbation in a patient matching an immune response signature observed in a healthy subject upon administration of the antigen, or if the antigen induced an immune perturbation in the patient that enhances the immune perturbation comprising the immune response signature of the disease, the induced immune perturbation can indicate or predict that the therapeutic agent is effective for the treatment of the disease in the patient. Alternatively, if the antigen has no impact or does not induce a desired immune perturbation in a patient in need of a treatment of a disease, it can suggest that the therapeutic agent is ineffective against the disease in the patient.

Accordingly, another aspect of the application relates to a method of predicting an effect of a therapeutic agent in a subject in need of a treatment of a disease, comprising:
(1) administering to the subject in need of the treatment an effective amount of a composition comprising an antigen known to elicit an immune perturbation in a healthy subject reflecting an immune response signature of the disease;
(2) collecting a biological sample from the subject in need of the treatment before and at multiple time points after the administering of step (1);
(3) measuring longitudinal changes from baseline (i.e., before the administering of step (1)) in immune cell populations and/or inflammatory mediators in the biological sample;
(4) comparing the measured longitudinal changes with the immune response signature of the disease to detect an effect of an immune perturbation induced by the antigen in the subject in need of the treatment; and
(5) predicting the effect of the therapeutic agent in the subject in need of the treatment based on the immune perturbation induced by the antigen.

The predicted effect of the therapeutic agent can be verified by administering the therapeutic agent to the subject. Thus, in one embodiment of the application, the method further comprises:
(1) administering a pharmaceutical composition comprising the therapeutic agent to the subject in need of the treatment of the disease, and
(2) evaluating the effect of the therapeutic agent in the subject in need of the treatment of the disease.

According to an embodiment of the application, the method further comprises measuring an impact of the therapeutic agent on the immune perturbation induced by the antigen in the subject in need of the treatment to thereby evaluate the effect of the therapeutic agent in the subject.

Technologies or methods known in the art can be used to identify key molecular mechanisms of innate and adaptive immunity during effector and memory stages of immune responses following vaccination to thereby obtain an immune response signature of a disease in a healthy subject administered with an antigen or vaccine (see, e.g., Haks et al., Front Immunol. 2017; 8: 1563. published online 2017 Nov. 15). For example, an extended multiplex transcriptomic profiling assay such as a dual-color Reverse-Transcription Multiplex Ligation-dependent Probe Amplification (dcRT-MLPA), can be used for bioprofiling the human immune response following vaccination against human infectious diseases, with particular emphasis on transcriptomic signatures of innate, adaptive, regulatory, inflammatory, and memory responses. Determination of optimal responses and dense kinetic transcriptomic response measurements will be helpful in optimizing novel immunization strategies. Transcriptomic analyses, such as global transcriptomic gene expression profiling (mRNA as well as micro-RNA), following vaccination with a vaccine, can be used to dissect novel mechanisms and correlates of immunity and safety, with particular emphasis on age and temporal changes following vaccination. Quantitative proteomic profiles using platform technologies, such as hydrogen deuterium exchange (HDX) coupled to mass spectrometry (MS), can be used to define functionally active epitopes and antibodies in polyclonal sera following vaccination. Antigen-specific memory T-cells and B-cells, and their ability to migrate to the correct tissue sites can also be analyzed using method known in the art, in view of the present disclosure. A combination of experimental approaches (antigenic stimulation, TCR deep sequencing and cloning of Th1, Th2, and Th17 memory subsets) can be used for the dissection of T-cell subset responses. Molecular mechanisms regulating T-cell trafficking at mucosal sites in health and disease, T-cell migration in response to chemokines (such as CCL20, CXCL10, and CXCL12) can also be assessed, as well as the capability of Th cells to reach mucosal niches to support cell maturation and functional activity, particularly in individuals experiencing chronic immune activation.

In one embodiment of the application, the induced immune perturbation in a healthy subject comprises a change in one or more immune pathways that are known to be dysregulated in a subject having the disease.

In another embodiment of the application, the induced immune perturbation in a healthy subject comprises a change in one or more immune cell populations that are known to be associated with the disease in a subject having the disease. Immune cell profiles can be studied and monitored using methods known in the art in view of the present disclosure.

In yet another embodiment of the application, the induced immune perturbation in a healthy subject comprises a change in one or more inflammatory mediators that are known to be associated with the disease in a subject having the disease. The level and/or activity of an inflammatory mediator can be studied and monitored using methods known in the art in view of the present disclosure.

In a further embodiment of the application, the induced immune perturbation in a healthy subject comprises a change in expression and/or activities of one or more biomarkers that are known to be associated with the disease in a subject having the disease. Methods known in the art can be used to study and monitor biomarkers of interest.

Any suitable antigen can be used to induce an immune perturbation reflecting an immune response signature of a disease in a healthy subject. Preferably, a vaccine or antigenic challenge is chosen based upon the following considerations: 1) approved used and safety record, 2) ability to activate multiple immune pathways at once or to stimulate specific immune cells and pathways of interest, and/or 3) ability to activate responses in the periphery or mucosal sites.

In one embodiment, a vaccine against a human infectious disease is used to induce an immune perturbation reflecting an immune response signature of a disease in a healthy subject. Examples of such vaccines include, but are not limited to, a vaccinia (smallpox) vaccine (e.g., Imvamune®/Imvanex®), a shingles vaccine (e.g., Shingrix®), a zoster vaccine (e.g., Zostavax®), and a tetanus vaccine.

In another embodiment, an endotoxin challenge, such as lipopolysaccharides (LPS) endotoxin, is used to induce an immune perturbation reflecting an immune response signature of a disease in a healthy subject. The endotoxin challenge has safely been used for decades to elicit immune responses that mimic those that occur during acute infection or in chronic inflammatory conditions (Dillingh et al, J of Inflammation 2014, Kiers et al, Sci Reports 2017).

In another embodiment, an antigenic skin challenge, such as CANDIN® (Candida albicans), is used to induce an immune perturbation reflecting an immune response signature of a disease in a healthy subject. Antigenic skin challenges have long been used to evaluate tissue-initiated antigen stimulation of localized and systemic soluble and cellular immune responses. These types of immune system challenges can provide insight regarding the expected range of normal immune responses in healthy individuals and as such serve as the basis for comparison to functional antigen specific immunity that may be altered in autoimmune diseases or by drug agents that target specific immune pathways (Lynn et al, J of Pharm and Exp Ther, 2004).

In one embodiment of the application, the biological sample collected from the healthy subject is a peripheral blood sample. Preferably, the biological sample comprises peripheral blood mononuclear cells (PBMCs). In another embodiment, the biological sample is a tissue sample. Any known method can be used to collect the biological sample from the healthy subject.

The biological samples are collected from the healthy subject before the administration of the antigen or vaccine, and at various time points after the administration. Preferably, the biological samples are collected from the healthy subject on day 0, before the administration of the antigen or vaccine, and on day 1, 3, 7, 14, 21, 28 up to 90 days after the administration of the antigen or vaccine.

Longitudinal post perturbation changes in cellular immune populations include, but are not limited to, changes in phenotype as measured by cell surface antigens associated with activation status and other functional attributes. Immune cells include cells of innate immune system as well as cells of adaptive immune system. Examples of immune cells include, but are not limited to, natural killer (NK) cells, eosinophils and dendritic cells (DC), neutrophil, macrophage, B cells and T cells.

Changes in inflammatory mediators include, but are not limited to, changes in the amount and/or activity of any biochemical mediators released during inflammation. Examples of inflammatory mediators include, but are not limited to, cytokines, chemokines, vasoactive amines (such as histamine and serotonin), kinins (such as bradykinin), eicosanoids (such as thromboxanes, leukotrienes, and prostaglandins), complement and complement-derived peptides, etc.

According to embodiments of the application, vaccines or antigens are used to induce an immune response in a healthy subject comprising an immune response signature reflective of the pathophysiology of a disease, and the effect of a therapeutic agent, preferably an immunotherapy, for treating a disease of interest is clinically evaluated by measuring an effect of the therapeutic agent on the response to the immune perturbation in the healthy subject. The inclusion of vaccines in early clinical studies on healthy subjects helps define individual baseline and immune response signatures to inform on response to the therapeutic agent (e.g., immunotherapy) in subjects in need of the treatment; inform on mechanism of action of the therapeutic agent; assess immune response thresholds; or support decision-making during early development phases prior to execution of larger clinical studies.

Thus, unlike most first-in-human studies that are intended to evaluate safety and pharmacokinetics of new drugs with less emphasis on pharmacodynamic drug effects, a method according to an embodiment of the application allows a first-in-human study of a therapeutic agent to also evaluate the pharmacodynamic effects of the therapeutic drug. For example, a doseresponse relationship of the therapeutic agent can be studied in a first-in-human study with healthy human subjects having an induced immune perturbation comprising an immune response signature reflective of the pathophysiology of the disease, prior to execution of further studies with patients or human subjects in need of a treatment of the disease.

Vaccinations or antigenic challenges have been used to establish an individual's immune response threshold or immunoscore (Tsang JS Trends in Immunology 36: 2015, Furman D and Davis MM, Vaccine 33:5271, 2015, Boyd, S D and Jackson, KJL Cell Host and Microbe, 17:301, 2015, Kaczorowski, K J et al PNAS, 2017). In view of the present disclosure, a vaccination or antigenic challenge can be used to induce in a healthy subject an immune perturbation comprising an immune response signature of a disease. This induced immune perturbation has potential predictive value for assessing responsiveness to an immune modulatory agent. In addition, vaccine or antigenic challenges can be practically utilized to evaluate biomarker responses in clinical trials of new immunomodulators, with a potential utility in assessing target engagement of the immunomodulator and collecting evidence of proof of mechanism (POM).

An immune perturbation can be measured and/or monitored using methods in the art in view of the present disclosure. The ability of an antigen or vaccine to induce or stimulate an immune response in an animal or human organism can be evaluated either in vitro or in vivo using a variety of assays which are standard in the art. For a general description of techniques available to evaluate the onset and activation of an immune response, see for example Coligan et al. (1992 and 1994, Current Protocols in Immunology; ed. J Wiley & Sons Inc, National Institute of Health). Measurement of cellular immunity can be performed by measurement of cytokine profiles secreted by activated effector cells including those derived from CD4+ and CD8+ T-cells (e.g. quantification of IL-10 or IFN gamma-producing cells by ELISPOT), by determination of the activation status of immune effector cells (e.g. T cell proliferation assays by a classical [³H] thymidine uptake or flow cytometry-based assays), by assaying for antigen-specific T lymphocytes in a sensitized subject (e.g. peptide-specific lysis in a cytotoxicity assay, etc.).

The ability of an antigen or vaccine to stimulate a cellular and/or a humoral response can be determined by antibody binding and/or competition in binding (see for example Harlow, 1989, Antibodies, Cold Spring Harbor Press). For example, titers of antibodies produced in response to administration of a composition providing an immunogen can be measured by enzyme-linked immunosorbent assay (ELISA). The immune responses can also be measured by neutralizing antibody assay, where a neutralization of a virus is defined as the loss of infectivity through reaction/inhibition/neutralization of the virus with specific antibody. The immune response can further be measured by Antibody-Dependent Cellular Phagocytosis (ADCP) Assay.

Any computational method can be used to monitor the changes in the measured immune responses over time. For example, Looper, a computational method is used to analyze measurement of in vivo immune response thresholds of an individual, thus provide a qualitative metric in which the likelihood of response and recovery to a particular immune perturbation can be assessed (Rath et al, Predicting position along a looping immune response trajectory, PLoS One, October 2018, the relevant content of which is incorporated herein by reference). According to an embodiment of the application, Looper is used to analyze an immune perturbation induced by vaccination in a healthy subject.

Several parameters can be considered when determining an individual's immune threshold response including static biomarkers (e.g. PD1 expression in situ, IFN signature) as well as functional responses to systemic immune challenges (e.g. anti-influenza infection or influenza vaccine response). In the field of systems immunology, the use of specific perturbations to measure the varied ranges of responses is being combined with static biomarker readouts to define a descriptive “immunoscore”. Thus, an individual can have a high or low score depending on baseline physiologic status or responsiveness. This immunoscore can then be subsequently considered for treatment stratification and treatment response monitoring, or clinical study enrollment of subjects in need to the treatment of a disease (Farsaci, B et al, Cancer Immunol Res, 4:755-765, 2016). According to an embodiment of the application, immune cell subsets within peripheral blood mononuclear cells in the healthy subject with the induced immune perturbation can be analyzed to determine if a “peripheral immunoscore” similar to that of Farsaci, B et al, could be included in the immune response signature of a disease.

According to an embodiment of the application, PBMCs are isolated from the subject pre and post administration of the antigen to assess cell populations and functional antigen specific immune responses. The PBMCs are divide by the types of subject (e.g. healthy vs patient), by immune challenges (e.g. with or without vaccination or types of vaccine administered), and by therapeutic agents evaluated (e.g., targeted immune modulator vs check point inhibitor). The PBMC samples can then be incorporated into future studies as controls or to interrogate other questions specific to the immune targeted by the therapeutic agent being evaluated in clinical trials. The healthy subject is administered with an antigen selected from the group consisting of: Immvamune® (smallpox vaccine), Shingrix® (Herpes zoster vaccine), endotoxin (E. coli LPS), Candida albicans, tetanus toxoid and Keyhole Limpet Hemocyanin (KLH). Panels of biomarkers are measured in PBMCs. The markers include, e.g., panels of immune cell specific phenotypic and functional markers that can be assessed by multiparameter flow cytometry or CyTof in addition to other types of functional assays that evaluate the production of antibodies and cytokines. Transcriptional data are also obtained including the use of single cell RNAseq from isolated PBMCs. One or more immune response signatures of a disease of interest can be identified based on the measurements of the markers, and comparison with markers known to be associated with the disease. An effect of a therapeutic agent on the disease is evaluated based on the measurements of the effect of the agent on the immune response signatures of a disease.

Methods of the application can be used for the clinical evaluation of any suitable therapeutic agent for treating a disease of interest in a subject in need thereof. Preferably, the therapeutic agent is an immune modulator. In some embodiments of the application, the therapeutic agent is a targeted immune modulator, such as one intended for the treatment of rheumatoid arthritis, juvenile idiopathic arthritis, ankylosing spondylitis, psoriatic arthritis, Crohn's disease, ulcerative colitis, or plaque psoriasis.

Also provided herein is a kit for clinical evaluation of a therapeutic agent. The kit comprises a composition comprising an antigen for use in inducing an immune perturbation comprising an immune response signature of a disease in a healthy subject, and a pharmaceutical composition comprising the therapeutic agent. The kit can also comprise one or more additional antigens or vaccines, adjuvants or immune stimulants. The kit can further contain instructions on using the composition comprising the antigen and the pharmaceutical composition in a clinical study according to a method of the application. Preferably, the composition comprising the antigen is selected from the group consisting of a vaccine against a human infectious disease, preferably a non-replicating smallpox vaccine, a herpes zoster vaccine, a non-live shingles vaccine, or a tetanus vaccine; an antigenic skin challenge, preferably a Candida albicans antigen; and an endotoxin, preferably a lipopolysaccharide (LPS). More preferably, the pharmaceutical composition comprises an immune modulator as an active ingredient, preferably a targeted immune modulator or a checkpoint inhibitor.

The following examples of the application are to further illustrate the nature of the application. It should be understood that the following examples do not limit the application and the scope of the application is to be determined by the appended claims.

### Example 1

### Multi-Level Analysis of Vaccine Induced Human Immune Responses

To determine the utility of vaccines or other antigenic/metabolic challenges as physiologic perturbations that can be used in future Phase 1 or Phase 2 human volunteer studies, an evaluation to confirm the time-course and range of vaccine/antigen-specific responses is conducted. This will also identify one or more immune response signatures for a disease of interest. The study will also permit assessment of the types of analysis tools that can be practically considered for use in future human clinical studies.

### Materials and Methods

This is a Phase 0, exploratory, single center study in healthy adult volunteers to evaluate the effect of 2 vaccines and 2 antigenic challenge agents on systemic immune responses and localized tissue, as applicable. Each subject will serve as their own control for the comparison of baseline cell population and inflammatory mediator responses. The end of the study will occur at the last sample collection time point defined for each cohort (up to 90 days) (see FIG. 3).

As shown in Table 1, commercially available vaccines, Imvamune®/Imvanex® (Bavarian Nordic) and Shingrix® (GSK) will be used. A commercially available allergen test solution will be used for the Candida albicans antigen skin injection and skin reaction induction. Commercially available LPS endotoxin will be used for the intravenous endotoxin challenge.

TABLE 1

| Co- | | | Sample | Sampling |
|---|---|---|---|---|
| hort | Intervention | Dose | collections | location |
| 1 | Imvamune ®/ | Single IM | Screening, D-1, | Peripheral blood |
| | Imvanex ® | injection | D1, 3, | |
| | | | 7, 14, 30, 90 | |
| 2 | Shingrix ® | Single IM | Screening, D-1, | Peripheral blood |
| | injection | 7, 14, 30, 90 | D1, 3, | |
| 3 | LPS endotoxin | Single | Screening, D-1, | Peripheral blood |
| | IVinjection | (Q2 hours × | D1 | |
| | | 12 hours), | D3, 30, 90 | |
| 4 | Candin ® | Single | Screening, D-1, | Peripheral blood, |
| | | intradermal | D1, 3, 7, 14 | injection site tissue |
| | | injection | | biopsy specimen |

The objectives and endpoints of the study are described in Table 2 below.

TABLE 2

| Objectives | Assessments |
|---|---|
| Primary | |
| Characterize the immune | Longitudinal change from baseline of immune cell |
| response in vivo using approved | Populations, associated changes in cell surface antigen |
| vaccines and antigen challenges | phenotype, activation status and the expression of |
| to stimulate the immune system. | inflammatory mediators measured in peripheral blood |
| | samples or tissues of healthy volunteers. |
| Secondary | |
| Assess intra-individual and inter- | Standard deviation of changes in the above-mentioned |
| individual variability in immune | endpoints within a participant and between participants in a |
| cellular and inflammatory | cohort. |
| mediator responses in a healthy | |
| individual or cohort of healthy | |
| individuals. | |
| Determine if there are baseline | Similarities in immune cell populations, inflammatory |
| characteristics of participants that | mediators, and their characteristics at baseline in a cohort or |
| predict responses to various | multiple cohorts will be measured by phenotypic and |
| challenges. | functional assays. |
| Exploratory | |
| Evaluate the feasibility of the use | Participant tolerance and compliance with a sleep and |
| of actigraphy devices in healthy | actigraphy device, and measurement of variability in |
| volunteers in a clinical study | responses as measured by digital sensors. |

### Results

Blood samples will be collected from participants for analysis of the immune response to the different challenge agents, including antigen-specific antibodies, cellular responses, and changes in inflammatory mediators. The analyses conducted on these samples are intended to provide insight on the impact on immunologic, hematologic and other organs systems, and/or general immune system function and the inter-individual variability in response. The detection and characterization of challenge agent-specific antibodies, cellular responses, and soluble inflammatory mediators will be measured using validated, specific, and sensitive immunoassay methods. Pharmacogenomic blood samples will be collected to allow for DNA extraction and analysis of genetic and epigenetic markers that are relevant to the antigen challenge specific immune pathways may be analyzed. The analysis can include complete genome-wide testing and/or targeted sequencing.

It is understood that the examples and embodiments described herein are for illustrative purposes only, and that changes could be made to the embodiments described above without departing from the broad inventive concept thereof. It is understood, therefore, that this invention is not limited to the particular embodiments disclosed, but it is intended to cover modifications within the spirit and scope of the invention as defined by the appended claims.

I/We claim:

1. A method of identifying an antigen that elicits an immune perturbation in a healthy subject reflecting an immune response signature of a disease, the method comprising:
(1) administering to the healthy subject an effective amount of a composition comprising an antigen;
(2) collecting a biological sample from the healthy subject before and at multiple time points after the administering of step (1);
(3) measuring longitudinal changes from baseline in immune cell populations and/or inflammatory mediators in the biological sample;
(4) comparing the measured longitudinal changes with the immune response signature of the disease to detect an effect of an immune perturbation induced by the antigen in the healthy subject; and
(5) identifying the antigen that elicits the immune perturbation in the healthy subject reflecting the immune response signature of the disease.

2. The method of claim 1 wherein the immune perturbation is selected from the group consisting of a change in one or more immune pathways that are dysregulated in a subject having the disease, a change in one or more immune cell populations associated with the disease in a subject having the disease, a change in one or more inflammatory mediators associated with the disease in a subject having the disease, and a change in expression and/or activities of one or more biomarkers associated with the disease in a subject having the disease.

3. A method of predicting an effect of a therapeutic agent, the method comprising:
(1) administering to a healthy subject an effective amount of a composition comprising an antigen known to elicit an immune perturbation in the healthy subject reflecting an immune response signature of a disease;
(2) administering a pharmaceutical composition comprising the therapeutic agent to the healthy subject having the immune perturbation;
(3) measuring an impact of the therapeutic agent on the immune perturbation in the healthy subject; and
(4) predicting the effect of the therapeutic agent in a subject in need of a treatment of the disease based on the impact measured in (3).

4. The method of claim 3, further comprises:
(1) administering the pharmaceutical composition comprising the therapeutic agent to the subject in need of the treatment of the disease, and
(2) evaluating the effect of the therapeutic agent in the subject in need of the treatment of the disease.

5. The method of claim 3, wherein the therapeutic agent decreases the immune perturbation in the healthy subject and is predicted to be effective for treating the disease in the subject in need of the treatment.

6. The method of claim 3 wherein the immune perturbation is selected from the group consisting of a change in one or more immune pathways that are dysregulated in a subject having the disease, a change in one or more immune cell populations associated with the disease in a subject having the disease, a change in one or more inflammatory mediators associated with the disease in a subject having the disease, and a change in expression and/or activities of one or more biomarkers associated with the disease in a subject having the disease.

7. A method of predicting an effect of a therapeutic agent, the method comprising:
(1) administering to a subject in need of a treatment of a disease an effective amount of a composition comprising an antigen known to elicit an immune perturbation in a healthy subject reflecting an immune response signature of the disease;
(2) collecting a biological sample from the subject in need of the treatment before and at multiple time points after the administering of step (1);
(3) measuring longitudinal changes from baseline in immune cell populations and/or inflammatory mediators in the biological sample;
(4) comparing the measured longitudinal changes with the immune response signature of the disease to detect an effect of an immune perturbation induced by the antigen in the subject in need of the treatment; and
(5) predicting the effect of the therapeutic agent in the subject in need of the treatment based on the immune perturbation induced by the antigen.

8. The method of claim 7, further comprising:
(1) administering a pharmaceutical composition comprising the therapeutic agent to the subject in need of the treatment of the disease, and
(2) evaluating the effect of the therapeutic agent in the subject in need of the treatment of the disease.

9. The method of claim 7, comprising measuring an impact of the therapeutic agent on the immune perturbation induced by the antigen in the subject in need of the treatment to thereby evaluate the effect of the therapeutic agent in the subject.

10. The method of claim 7 wherein the immune perturbation is selected from the group consisting of a change in one or more immune pathways that are dysregulated in a subject having the disease, a change in one or more immune cell populations associated with the disease in a subject having the disease, a change in one or more inflammatory mediators associated with the disease in a subject having the disease, and a change in expression and/or activities of one or more biomarkers associated with the disease in a subject having the disease.

11. The method of claim 3 wherein the method is the first-in-human study of the therapeutic agent, preferably the effect of the therapeutic agent comprises a pharmacodynamic effect of the therapeutic agent in human subjects, more preferably, the pharmacodynamic effect comprises a dosage-response relationship of the therapeutic agent.

12. The method of claim 1, wherein the immune response signature of the disease overlaps with at least one selected from the group consisting of: (1) an immune response that occurs during acute infection or chronic inflammatory conditions associated with the disease; (2) localized and/or systemic soluble and cellular immune responses associated with the disease; and (3) metabolic responses to dietary components associated with the disease.

13. The method of claim 3, wherein the immune response signature of the disease overlaps with at least one selected from the group consisting of: (1) an immune response that occurs during acute infection or chronic inflammatory conditions associated with the disease; (2) localized and/or systemic soluble and cellular immune responses associated with the disease; and (3) metabolic responses to dietary components associated with the disease.

14. The method of claim 7, wherein the immune response signature of the disease overlaps with at least one selected from the group consisting of: (1) an immune response that occurs during acute infection or chronic inflammatory conditions associated with the disease; (2) localized and/or systemic soluble and cellular immune responses associated with the disease; and (3) metabolic responses to dietary components associated with the disease.

15. The method of claim 1, wherein the antigen is a vaccine against a human infectious disease, preferably the composition is selected from the group consisting of a non-replicating smallpox vaccine, a herpes zoster vaccine, a non-live shingles vaccine, and a tetanus vaccine.

16. The method of claim 3, wherein the composition comprising the antigen is a vaccine against a human infectious disease, preferably the composition is selected from the group consisting of a non-replicating smallpox vaccine, a herpes zoster vaccine, a non-live shingles vaccine, and a tetanus vaccine.

17. The method of claim 7, wherein the composition comprising the antigen is a vaccine against a human infectious disease, preferably the composition is selected from the group consisting of a non-replicating smallpox vaccine, a herpes zoster vaccine, a non-live shingles vaccine, and a tetanus vaccine.

18. The method of claim 1, wherein the antigen is selected from group consisting of an antigenic skin challenge and an endotoxin.

19. A kit for clinical evaluation of a therapeutic agent, comprising:
a. a composition comprising an antigen for use in inducing an immune perturbation comprising an immune response signature of a disease in a healthy subject; and
b. a pharmaceutical composition comprising a therapeutic agent.

20. The kit of claim 19, wherein the composition comprising the antigen is selected from the group consisting of a vaccine against a human infectious disease, preferably a non-replicating smallpox vaccine, a herpes zoster vaccine, a non-live shingles vaccine, or a tetanus vaccine; an antigenic skin challenge, preferably a Candida albicans antigen; and an endotoxin, preferably a lipopolysaccharide (LPS) endotoxin.
